(19) United States
(12) Patent Application Publication
(10) Pub. No.: US 2007/0259335 A1
(43) Pub. Date: Nov. 8, 2007

(54) Method for Predicting De Novo Biomacromolecule Crystallization Conditions and for Crystallization of the Same

(76) Inventors: Xiang-Yang Liu, Singapore (SG); Yan Wei Jia, Singapore (SG)

Correspondence Address: FISH & RICHARDSON, PC, P.O. BOX 1022, MINNEAPOLIS, MN, 55440-1022, US

(21) Appl. No.: 10/590,536

(22) PCT Filed: Feb. 21, 2005
(86) PCT No.: PCT/SG05/00051

§ 371 Date: May 3, 2007

Related U.S. Application Data
(60) Provisional application No. 60/547,730, filed on Feb. 25, 2004.

(51) Int. Cl. G01N 33/68; C12Q 1/70; G01N 33/50

(52) U.S. Cl. 435/5; 436/86; 436/94

(57) ABSTRACT

A method de novo is provided for predicting crystallization conditions and for crystallizing biomacromolecules, in particular proteins. The method provides a simple, quick and precise approach in determining the biomacromolecule solubility in different solutions, as well as the boundary between crystallization and aggregation. Because the method relies only on monitoring the assembly behavior of the biomacromolecule at the surface of a solution, it has general applicability and requires a relatively short amount of time to provide results that are reliable. Because there is no need to first crystallize the biomacromolecule, small amounts of protein are sufficient. Because the method works by measuring the surface tension or pressure of the surface of the biomacromolecule solution, it is easy, precise and quick. Furthermore it is cost-effective in requiring simple and inexpensive equipment.

### FIELD OF INVENTION

This invention relates to a de novo method of predicting crystallization conditions. In particular, the invention relates to a method of predicting the conditions for crystallizing biomacromolecules from solution, and more specifically for crystallizing proteins from solution.

### BACKGROUND

In most cases of protein crystallization experiments, the final product is not a single crystal but amorphous aggregation. To predict the likelihood of the formation of either a crystal or an amorphous aggregation, the second virial coefficient B₂₂ is customarily employed by many groups [1-5]. Serving as an indicator of intermolecular interactions, B₂₂ is positive when these interactions are repulsive, and negative when these interactions are attractive. As a consequence, a necessary condition for crystallization is obtained when the second virial coefficient B₂₂ lies in a so-called “crystallization window”: −8×10⁻⁴<B₂₂<−2×10⁻⁴ ml mol/g².

The criterion based on the second virial coefficient has its advantages, as it gives a discriminating response. However this criterion does not work in some cases in which B₂₂ could well lie within the crystallization window, but the experiment gives amorphous aggregation. One of the reasons for this failure is that the criterion provided by the second virial coefficient takes into account only the interactions between the biomacromolecules. However, such intermolecular interactions determine biomacromolecule crystallization only partially. Apart from this, the B₂₂ criterion only attempts to determine the solvent conditions for which crystallization rather than amorphous aggregation would take place. However, for biomacromolecule crystallization to be possible in the first place, an additional condition must be satisfied, namely that the biomacromolecule concentration must exceed its equilibrium value in the context of the applied solvent conditions. The B₂₂ criterion does not address this problem, and gives no information whatsoever on the critical equilibrium condition of the biomacromolecule.

The crystallization of biomacromolecules involves a nucleation and growth process, determined to a large extent by kinetics. Kinetics refers to the way biomacromolecules move in a solution, the rate at which they are transported, and the way they are incorporated in the biomacromolecule crystals at the crystal surface. The crystallization window provided by the second virial coefficient disregards kinetic and other factors, that are unrelated to intermolecular interactions but nevertheless largely influence crystallization.

Alternative methods have been developed to circumvent the drawbacks presented by the use of the second virial coefficient in attempting to predict crystallization. These methods, the so-called high throughput screening methods, are solely empirical involving a large number of solution matrices. These methods are costly, cumbersome, and time-consuming; they require large investments in expensive robots. As protein crystallization is normally a lengthy process and can be affected by about 20 different parameters, these methods have in many cases no general applicability and suffer from a low success rate.

What is needed is a de novo method offering a twofold advantage above the prior art. Firstly, such a method should determine the equilibrium biomacromolecule concentration, so that the values of the biomacromolecule concentration used in the process of crystallization in the prevailing experimental conditions can be restricted to values above the equilibrium value. Secondly, such a method should predict more reliably the crystallization conditions referring to the solvent experimental parameters. Finally, such a method should have a more general applicability and be simple, easy to apply, not wasteful on the biomacromolecule or the protein. It should not require complex equipment, or rely on heavy investments for its application, and it should be amenable to being utilized by both the institutional and industrial establishments, while at the same time offering substantial automation advantages, and being much less costly than the methods recited in the prior art. Such a de novo method does not exist in the prior art.

### REFERENCES

R. A. Curtis, J. M. Prausnitz et al., Protein-protein and protein-salt interactions in aqueous protein solutions containing concentrated electrolytes, Biotechnol. Bioeng, 57: 11-21(1998).
Y. C. Chiew, D. Kuehner et al., Molecular thermodynamics for salt-induced protein precipitation, AIChE J. 41: 2150-2159 (1995).
A. George and W. W. Wilson, Predicting protein crystallization from a dilute solution property, Acta Crystallogr. D. 50: 361-365 (1994).
B. L. Neal and D. Asthagiri et al., Why is the osmotic second virial coefficient related to protein crystallization? J. Crystal Growth, 196: 377-387 (1999).
F. Bonnete and S. Finet et al., Second virial coefficient variations with lysozyme crystallization conditions, J. Crystal Growth, 196: 403-414 (1999).
E. Tornberg, The application of the drop volume technique to measurement of the adsorption of protein at interfaces, J. Colloid Interface Sci. 64(3): 391-402 (1978).
D. E. Graham and M. C. Phillips, Protein at liquid interfaces I: kinetics of adsorption and surface denaturation, J. Colloid Interface Sci. 70(3): 403-417 (1979).
M. Subirade et al., Effect of dissociated and conformational changes on the surface behavior of pea legumin, J. Colloid Interface Sci. 152(2): 442-454 (1992).

### SUMMARY

A de novo method for predicting crystallization conditions for biomacromolecules is provided, giving a more reliable prediction criterion than has been possible thus far. Unlike the second virial coefficient B₂₂ used to predict crystallization conditions based solely on intermolecular interactions, the present invention combines information on both the intermolecular interactions and kinetic effects to prescribe crystallization conditions. The improvement above the empirical methods available in the prior art and the prediction methods provided by calculating the second virial coefficient, is due to the incorporation of the kinetic effects that largely determine biomacromolecule crystallization, and offers the following advantages: (1) it establishes an equilibrium macromolecule concentration that should be exceeded by the applied macromolecule concentration used for crystallization; (2) only small amounts of protein are required; (3) the measurements are much quicker than either through use of the B₂₂ approach or through use of the empirical screening methods; (4) the simplicity of the required measurement of the assembly parameter (e.g. surface tension) of the biomacromolecule solution facilitates the handling in an ordinary laboratory.

One object of the present invention is to identify the phase boundary between a crystal phase and a liquid phase by determining the biomacromolecule solubility, under prevailing experimental conditions. Another object of the invention is to identify the boundary between a crystallization and an aggregation under prevailing experimental conditions.

Therefore in accordance with a first aspect of the invention there is disclosed:

A method for predicting a crystal equilibrium condition for biomacromolecule crystallization and for crystallizing a biomacromolecule, comprising

setting up at least one biomacromolecule solubility experiment comprising the steps of

a) preparing a solution of the biomacromolecule in a solvent, the solution having a biomacromolecule concentration,
b) selecting a variable quantity,
c) selecting an assembly parameter,
d) monitoring a response of the assembly parameter while varying the variable quantity in a suitable way so that the response exhibits a transition,
e) obtaining an equilibrium biomacromolecule concentration based on the transition,
f) defining a crystal equilibrium condition according to which a biomacromolecule crystallization concentration exceeds the equilibrium biomacromolecule concentration,
   and crystallizing the biomacromolecule.

In accordance with a second aspect of the invention there is disclosed:

A method for predicting an aggregation boundary condition for biomacromolecule crystallization and for crystallizing a biomacromolecule, comprising

setting up at least one aggregation boundary condition experiment comprising

a) preparing a solution of the biomacromolecule,
b) selecting a variable quantity,
c) selecting an assembly parameter,
d) measuring the assembly parameter at different times,
e) registering an equilibrium assembly parameter
f) deriving a crystallization coefficient from the equilibrium assembly parameter, the crystallization coefficient being associated with the variable quantity,
g) using an aggregation indicator to define an aggregation boundary condition for the biomacromolecule, the aggregation boundary condition prescribing that an aggregation occurs when the crystallization coefficient associated with the variable quantity is larger than the aggregation indicator,
   and crystallizing the biomacromolecule.

### BRIEF DESCRIPTION OF THE DRAWINGS

Embodiments of the present invention are illustrated by the following drawings:

FIG. 1 is an illustration of predictive conditions;

FIG. 1A is an illustration of a crystal equilibrium condition, leading to an equilibrium biomacromolecule concentration;

FIG. 1B is an illustration of an aggregation boundary condition, leading to a crystallization coefficient;

FIG. 2 is an illustration of a crystallization process from a solution of biomacromolecules, involving a transition from a liquid phase to an orderly solid phase, that is a crystal;

FIG. 3 is an illustration of an amorphous aggregation process involving the transition from the liquid phase to a disorderly solid phase;

FIG. 4 is an illustration of analogous processes to the crystallization and the amorphous aggregation that take place in the solution;

FIG. 5 is an illustration of a response of an assembly parameter to a tendency of the biomacromolecules to assembly;

FIG. 6 is an illustration of the way a solubility curve relates to the crystal equilibrium condition.

FIG. 7 shows plots of the assembly parameter against time, when the solution of biomacromolecules includes a protein lysozyme and a salt sodium chloride;

FIG. 8 shows plots of the assembly parameter against the square root of the time;

FIG. 9 shows plots of the logarithm of the time derivative of the assembly parameter;

FIG. 10 is an illustration of a time-dependence profile of a macromolecule behavior;

FIG. 11 shows plots of a diffusion time, a penetration time and a rearrangement time;

FIG. 12 shows a histogram illustrating how crystallization coefficients relate to the aggregation boundary condition by prescribing an aggregation indicator;

FIG. 13 shows a plot of the assembly parameter against time, when the solution of biomacromolecules includes the protein lysozyme and a salt ammonium sulfate;

FIG. 14 shows a plot of the assembly parameter against time, when the solution of biomacromolecules includes a protein concalavine A and the salt ammonium sulfate; and

FIG. 15 shows plots of the assembly parameter against time, when the solution of biomacromolecules includes a protein bovine serum albumin (BSA) and the salt ammonium sulfate.

### DETAILED DESCRIPTION

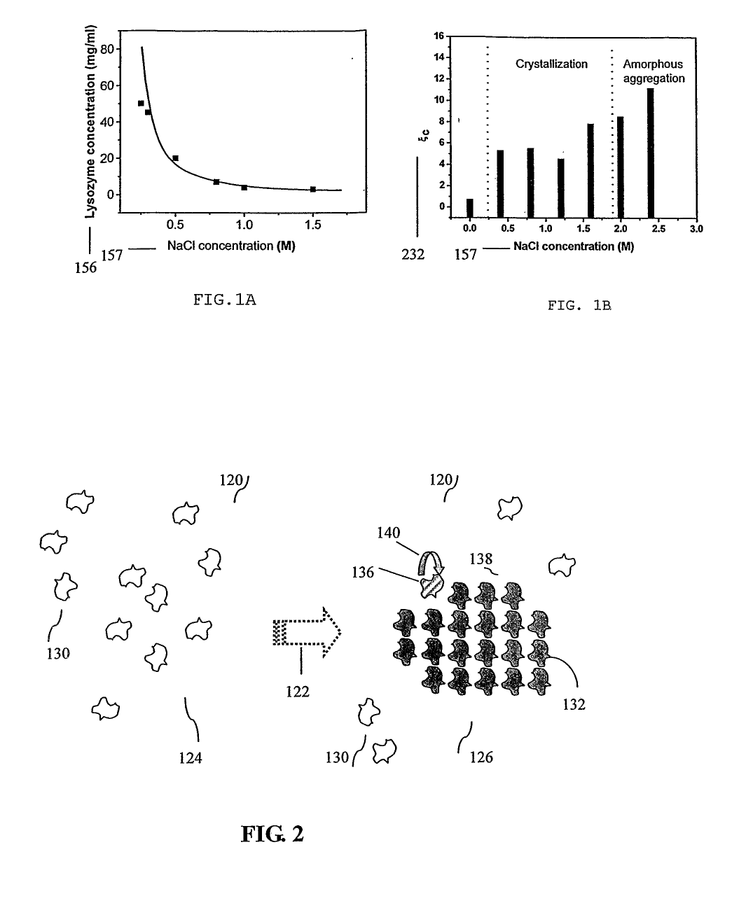

The primary features of a first embodiment of the invention of a method of predicting biomacromolecule crystallization conditions and for crystallizing biomacromolecules are provided hereinafter with reference to FIG. 1A in FIGS. 2-6. The primary features of a second embodiment of the invention are provided hereinafter with reference to FIG. 1B in FIGS. 2-5 and FIGS. 7-15. In the first embodiment, a crystal equilibrium condition in FIG. 1A is expressed by means of a macromolecule solubility curve serving as a boundary separating two regions of experimental parameter values: a region where crystallization can occur, and a region where crystallization cannot occur.

The method described in the first embodiment establishes a biomacromolecule equilibrium concentration in the context of the applied experimental conditions. The biomacromolecule concentration to be used for crystallization must exceed the obtained equilibrium value. In a second embodiment, an aggregation boundary condition in FIG. 1B is expressed by means of a window of experimental parameter values above which the amorphous aggregation is likely to occur. The terms “aggregation” and “amorphous aggregation” are used interchangeably.

Theoretical Introduction

In attempts to crystallize biomacromolecules from a solution, it is desirable to obtain as much as possible single crystals with as few defects as possible, and to avoid amorphous aggregations of molecules, since amorphous aggregates are not crystals. The present invention takes advantage of the property of biomacromolecules to have mixed hydrophobic and hydrophilic regions. This property results in a tendency for these molecules to assembly either in the bulk or at the surface of the solution. In this disclosure, the surface of the solution can be adjacent to another material or to empty space, and hence the surface can be in contact with a solid or with a liquid or with a gas, that is usually air. The surface of the solution has a surface tension and a surface pressure, which terms in this case include an interfacial tension or an interfacial pressure.

It is possible to define one or more assembly parameters that reach a critical response as increasingly more molecules participate in assembly formation. For example, the tendency of biomacromolecules to assembly in a solution can be monitored by taking density, conductivity, detergency, osmotic pressure, surface tension or surface pressure measurements of the solution.

Biomacromolecule crystallization conditions are reflected in the tendency of biomacromolecules to assembly. The crystallization conditions and the assembly characteristics of biomacromolecules are governed by both the intermolecular forces and the kinetic effects [6-8], depending on the experimental situation. The present invention prescribes two procedures of measuring the assembly parameters in solution so as to determine reliable crystallization conditions without the prior need to carry out crystallization experiments. Without limitation to the scope of the invention, the present examples illustrating the invention show that the disclosed method is particularly applicable when the biomacromolecule is not prone to severe unfolding at the surface of the solution or at an air/solution interface.

Application

In FIG. 2 a crystallization process from the solution 120 comprising biomacromolecules 130 involves a transition 122 from a liquid phase 124 to an orderly solid phase, that is a crystal 126, wherein solid-phase biomacromolecules 132 are regularly arrayed and regularly oriented. The biomacromolecules 130 in the liquid phase 124, become adsorbed biomacromolecules 136 on a surface 138 of the crystal 126, and subsequently they acquire a suitable orientation 140 in order to become incorporated as in the crystal 126 as the solid-phase biomacromolecules 132. The crystallization occurs because a transport time, being an amount of time required for the biomacromolecules 130 in the solution 120 to reach the surface 138 of the crystal 126, otherwise known as a diffusion time t_{diff}, is longer than an integration time tᵢₙₜ, wherein the integration time is an amount of time required for the adsorbed biomacromolecules 136 to become the solid-phase biomacromolecules 132, and it includes an amount of time required for their rearrangement.

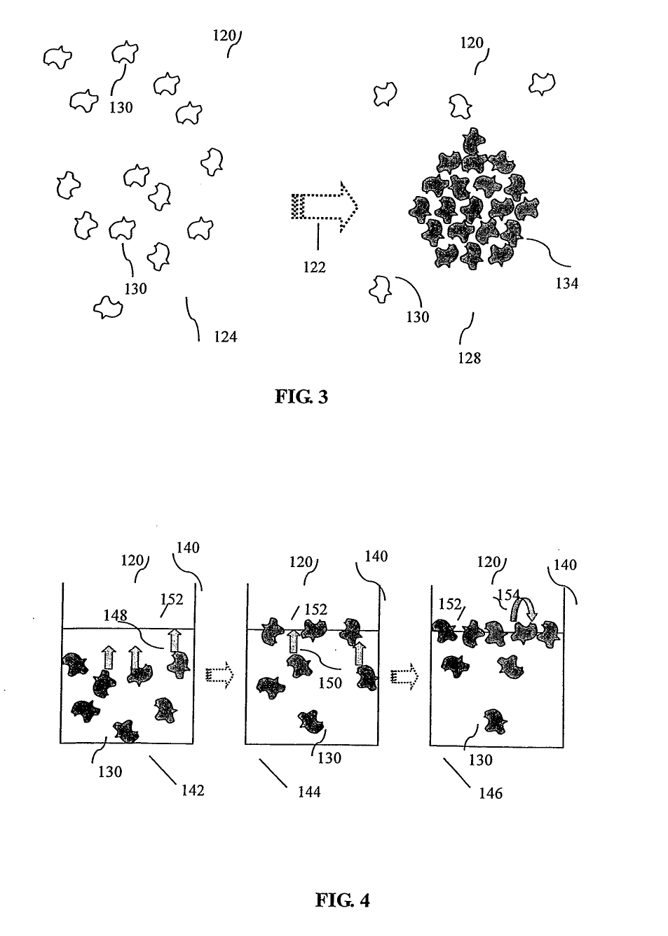

In FIG. 3 on the other hand, the amorphous aggregation involves the transition 122 from the liquid phase 124 to a disorderly solid phase 128, wherein aggregate biomacromolecules 134 have irregular positions and irregular orientations. The amorphous aggregation occurs because the diffusion time t_{diff} required for the biomacromolecules 130 in the solution 120 to reach the solid phase 128 is shorter than the time required for their rearrangement and integration.

In FIGS. 2 and 3 the solution 120 has an appropriate temperature and an appropriate pH. In the invention there is no restriction on the appropriate temperature, which is usually taken to be around a room temperature. A buffering material may be introduced in the solution 120 to maintain a desirable level of the pH. The solution 120 can also comprise additives like small or large organic molecules or salt. By way of example, the biomacromolecule 130 in FIGS. 2 and 3 is a protein, more specifically lysozyme (about 14.3 kDalton) or concalavine A (about 23 kdalton), and the additive is a salt, more specifically sodium chloride or ammonium sulfate.

FIG. 4 illustrates that processes analogous to the crystallization or the amorphous aggregation take place in the solution 120 containing the biomacromolecules 130. The solution 120 is placed in a container 140, and the solution has a surface 152, which in this application is adjacent to air. During a diffusion step 142, the biomacromolecules 130 take an average amount of time, that is the diffusion time t_{diff}, to diffuse 148 towards the surface 152. Subsequently, during a penetration step 144, the biomacromolecules 130 take an average amount of time, that is a penetration time tₚₑₙ, to penetrate 150 the surface 152. Subsequently, during a rearrangement step 146, the biomacromolecules 130, that penetrated 150 the surface 152 take an average amount of time, that is a rearrangement time tₐᵣᵣ, to undergo a configurational rearrangement 154 and finally become integrated in the surface 152. Thus the integration time tᵢₙₜ is the sum of the penetration time and the rearrangement time, tᵢₙₜ=tₚₑₙ+tₐᵣᵣ, because it equals an amount of time required to complete the penetration step 144 and the rearrangement step 146.

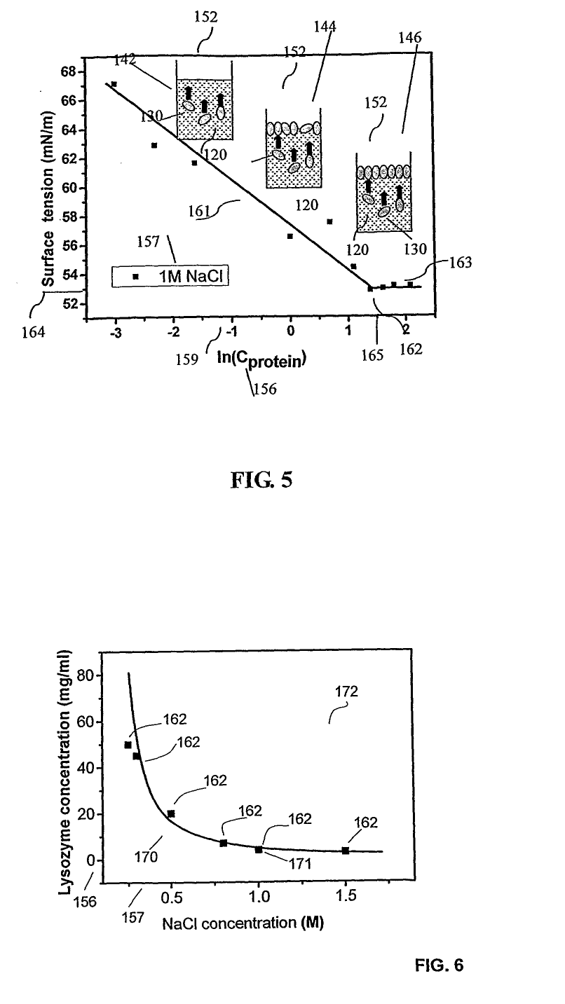

In FIG. 5 the biomacromolecules 130 have a biomacromolecule concentration 156, and the additives have an additive concentration 157. Without limitation to the scope of the invention, in the experimental runs of the first embodiment of the invention, the biomacromolecule concentration 156 is taken, e.g. 4 mg/ml lysozyme, while the additive (e.g. salt) concentration 157 is allowed to vary. In some other experimental runs of the first embodiment of the invention, the additive concentration 157 is taken, e.g. 1 M (as in FIG. 5) or 2 M salt, while the biomacromolecule concentration 156 is allowed to vary.

Referring to FIGS. 5 and 6, in the first embodiment of the invention, measurements of an assembly parameter 164 (for example a surface tension or a surface pressure) with respect to a variable quantity 158 (for example the biomacromolecule concentration 156, the additive concentration 157, the pH or the temperature), are utilized to express the crystal equilibrium condition. According to that condition, as will be explained further on, in FIG. 1A (FIG. 6), the crystallization is expected in a region 172 delineated by (and lying above and to the right of) a biomacromolecule solubility curve 170.

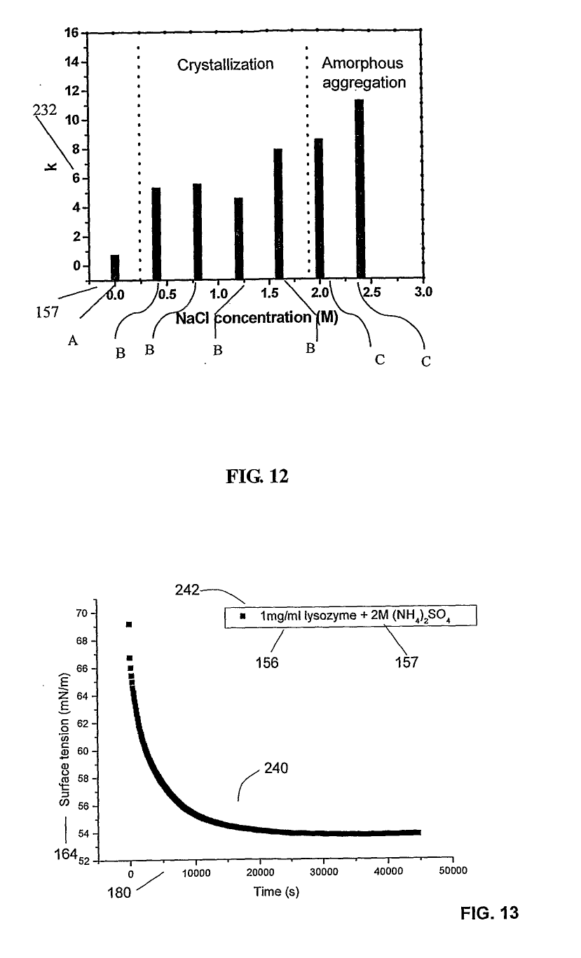

In the second embodiment of the invention, a probability that the aggregation (FIG. 3) will dominate above the crystallization (FIG. 2), increases when the integration time tᵢₙₜ is substantially longer than the diffusion time t_{diff}. That probability can be quantified as illustrated in FIG. 1B (FIG. 12), by means of a crystallization coefficient 232 k_{cryst}=tᵢₙₜ/t_{diff} defined as a ratio of the integration time to the diffusion time. Good quality crystallization occurs when k_{cryst} is in a suitable range. The higher the value of k_{cryst}, the higher the probability that the amorphous aggregation will occur. When above k_{cryst}, the solution 120 containing the biomacromolecule 130 exists in a so-called “steady state”, in which neither the crystallization (126 in FIG. 2) nor the aggregation (128 in FIG. 3) can take place. The aggregation indicator resulting from the crystallization coefficient should therefore certainly lie well above k_{cryst}=1; the likely reason for this is that if k_{cryst}<1, the diffusion step 142 of the biomacromolecules 130 in the solution 120 lasts such a long time, that it becomes difficult for the biomacromoles 130 to come in contact and group with one another. In practice however, in our experimental applications, it turns out that a realistic minimum value for k_{cryst}, allowing biomacromoles 130 to come in contact and group lies around 4 or 4.5.

Because the crystallization coefficient 232 is a dimensionless ratio, it is expected to have general applicability for most biomacromolecules. The method is illustrated in this experiment by choosing as examples the protein lysozyme to serve as a model system for the biomacromolecule crystallization, as well as the protein concanavalin A. It is therefore expected that the aggregation indicator obtained from the crystallization coefficient derived for the proteins lysozyme and concanavalin A will serve as a standard criterion to define the aggregation boundary condition for the crystallization of most biomacromolecules.

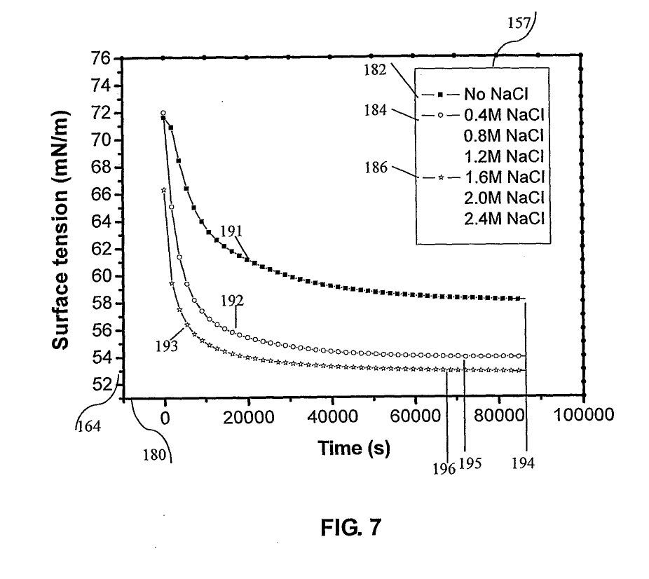

In the second embodiment of the invention, the solution 120 in FIGS. 4 and 5 is fairly dilute. Without limitation to the scope of the invention, in some experimental runs the biomacromolecule concentration is taken 156, 158 in FIGS. 4 and 5 is taken in the range 0.01 mg/ml to about 1.2 mg/ml. Measurements of the diffusion time t_{diff}, the penetration time tₚₑₙ and the rearrangement time tₐᵣᵣ are used to calculate the crystallization coefficient 232 k_{cryst}=tᵢₙₜ/t_{diff} which in turn is used to express the aggregation boundary condition, as explained further below with reference to FIGS. 7-15.

### First Embodiment

### Crystal Equilibrium Condition

In the first embodiment of the invention, in FIGS. 4-6, the crystal equilibrium condition is determined by constructing the biomacromolecule solubility curve 170.

FIG. 5 illustrates a response of the assembly parameter 164 to the activity of the biomacromolecules 130 in the solution 120. A suitable assembly parameter 164 for this application is the surface tension or the surface pressure, and in this case it is taken to be the surface tension, that can be measured with a tensiometer. The surface pressure p is related to the surface tension s by the equation: p=s−s₀, where so is the surface tension of the solvent in the absence of biomacromolecules. A suitable variable quantity 158 for this application is the biomacromolecule concentration 156 or the additive concentration 157, the pH, or the temperature. When a suitable variable quantity 158 has been selected, it can be made to vary in order to sample the response of the assembly parameter 164. In this case the variable quantity 158 is taken as the biomacromolecule concentration 156, whereas the additive concentration 157 is taken to be 1 M salt, e.g. NaCl.

FIG. 5 shows a plot of the assembly parameter 164 (the surface tension) against the logarithm 159 of the variable quantity 158 (the biomacromolecule concentration 156), according to which the assembly parameter 164 decreases as the variable quantity 158 is gradually increased. This is illustrated by the diffusion step 142, followed by the penetration step 144, subsequently resulting in completion of the rearrangement step 146. Upon completion of the rearrangement step 146, a complete layer of rearranged biomacromolecules has been assembled at the surface 152, allowing no space for any more biomacromolecules 130 to penetrate the surface 152. Therefore a further increase of the variable quantity 158, being in this experiment the biomacromolecule concentration, cannot cause a further decrease in the assembly parameter 164, being in this experiment the surface tension, but will instead result in accumulation of biomacromolecules 130 in the bulk of the solution 120.

FIG. 5 illustrates a biomacromolecule solubility experiment. Thus in FIG. 5 as more and more biomacromolecules 130 tend towards assembly formation, the response of the assembly parameter 164 shows a transition 162 between a changing response 161 and a substantially constant response 163 of the assembly parameter 164. The transition 162 is associated not only with a critical response of the assembly parameter 164, but also with a critical magnitude 165 of the variable quantity 158. In this case the critical response of the assembly parameter 164 is equal to the surface tension of 53 mN/m. The critical magnitude 165 of the variable quantity 158 is equal to the lysozyme concentration of 4 mg/ml; this corresponds to the logarithm 159 of the variable quantity 158 equal to 1.4, when the additive concentration 157 is equal to 1 M NaCl.

Because in this particular example the assembly parameter 164 is the surface tension, it decreases as the variable quantity 158, that is the biomacromolecule concentration 156, is increased, and hence the critical response of the assembly parameter 164 is substantially minimal. The crystal equilibrium condition can be expressed by means of the critical magnitude 165 of the variable quantity 158: when the temperature and pH are held at their predetermined values, and when the salt concentration is held at 1 M NaCl, then the crystallization cannot occur for values of the lysozyme concentration 156 falling below the equilibrium biomacromolecule concentration, the value of which in the prevailing experimental conditions is 4 mg/ml. A similar critical behavior is observed when the additive concentration 157 is allowed to vary, while the biomacromolecule concentration 156 is held constant at 4 mg/ml lysozyme (drawing not shown).

The aforementioned behavior should not be construed to be typical of a general case covered by the scope of the invention in which the transition 162 refers to the changing response and the substantially stable or the substantially unchanging response, considering that the changing response need not imply a purely decreasing or a purely increasing response.

FIG. 6 illustrates how the biomacromolecule solubility curve 170 can be constructed in order to express the crystal equilibrium condition. The solubility curve 170 follows when two of the following quantities: the biomacromolecule concentration 156, the additive concentration 157, the pH and the temperature, are allowed to form a pair of the variable quantities. In the example of FIG. 6 the biomacromolecule concentration 156 and the additive concentration 157 form the pair of the variable quantities, while the pH is kept fixed. A series of transitions 162 is obtained, each transition associated with the critical response of the assembly parameter 164. In the particular experiment described here, six transitions 162 were measured and employed in order to construct the solubility curve 170. For example, a point 171 on the solubility curve 170 indicates the following pair of the variable quantities: the biomacromolecule concentration 156 is 4 mg/ml lysozyme while the additive concentration 157 is around 1 M NaCl.

The corresponding crystal equilibrium condition follows by specifying that the crystallization can occur in a crystallization region 172 in which either one of the pair of the variable quantities assumes values at or above the corresponding critical magnitude 165 on the solubility curve 170. Therefore the crystal equilibrium condition as illustrated in FIG. 6 is obtained by recording a series of the critical magnitudes 165 of the variable quantity and the corresponding critical response of the assembly parameter 164, as illustrated in FIG. 5.

When the biomacromolecule concentration 156 is used as a variable quantity, the critical magnitude 165 is the equilibrium biomacromolecule concentration. When some other parameter is used as a variable quantity, e.g. one of the additive (salt) concentration 156 or the pH, or the temperature, the equilibrium biomacromolecule concentration is, in the context of the employed experimental conditions, given by the applied biomacromolecule concentration 156. In either case the crystal equilibrium condition prescribes that for crystallization to take place, the crystallization biomacromolecule concentration must exceed the equilibrium biomacromolecule concentration resulting from the biomacromolecule solubility experiment.

For the measurement of the points on the solubility curve the Wilhelmy plate method is employed using a K14 Kruss tensiometer, according to the following steps.

1. A buffer solution is prepared at the predetermined pH. A biomacromolecule stock solution is prepared by dissolving the biomacromolecule in the buffer solution. An additive stock solution is prepared by dissolving the additive in the buffer solution.

2. In FIG. 5, various solutions 120 are prepared at the predetermined temperature, usually room temperature, having the additive concentration 157 and different biomacromolecule concentrations 156 starting from 0 and increasing as long as the solutions 120 maintain a clear appearance. The solutions 120 are kept at the predetermined temperature for a few hours. The surface tension or surface pressure 164 of each solution 120 is measured by putting it into the Kruss tensiometer until the surface tension or pressure reaches a constant value. The chamber of the tensiometer is saturated with pure water vapor to maintain a uniform humidity, and the solutions 120 are kept at the predetermined temperature.

The surface tension or surface pressure 164 is recorded and plotted against the biomacromolecule concentration 156. The critical magnitude 165 of the biomacromolecule concentration 156 occurring at the critical point 162 is registered. It corresponds to the surface tension or surface pressure 164, undergoing the transition 162 from the changing response 161 to the substantially constant response 163 as the biomacromolecule concentration 156 increases. The critical point 162 determined in this way is a solubility value of the biomacromolecule at the additive concentration 157.

Step 2 is repeated to obtain solubility points at different additive concentrations 157. Steps 1 and 2 are repeated to find the solubility values at different additive concentration 157 and different pH.

### Second Embodiment of the Invention

### Aggregation Boundary Condition

In the second embodiment of the invention, the measurement of the aggregation boundary condition is carried out, as illustrated in FIGS. 4 and 5, by the following steps.

1. The buffer solution is prepared at the predetermined pH. The biomacromolecule stock solution is prepared by dissolving the biomacromolecule in the buffer solution. The additive stock solution is prepared by dissolving the additive in the buffer solution.

2. Various solutions 120 are prepared having the biomacromolecule concentration 156 and different additive concentrations 157. As already stated, the biomacromolecule solution 120 in FIG. 4, used in the second embodiment for obtaining the aggregation boundary condition, should be fairly dilute. In the particular experiments described here the protein concentrations 130 are e.g. 0.01, 0.1 or 1.0 mg/ml. Immediately after mixing each of the solutions 120, the surface tension is measured with respect to time and recorded. The measurement is stopped when the surface tension in each solution reaches a constant value. The obtained data are analyzed according to the steps outlined in FIGS. 7-15 described below.

Step 2 is repeated for different pH.

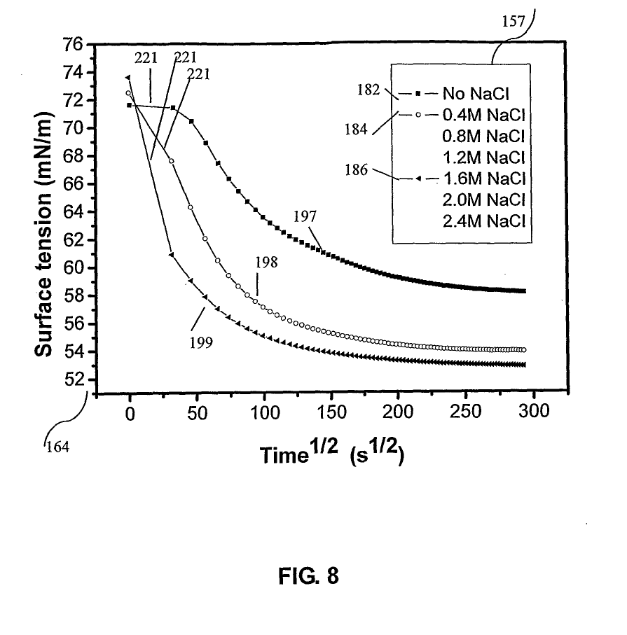

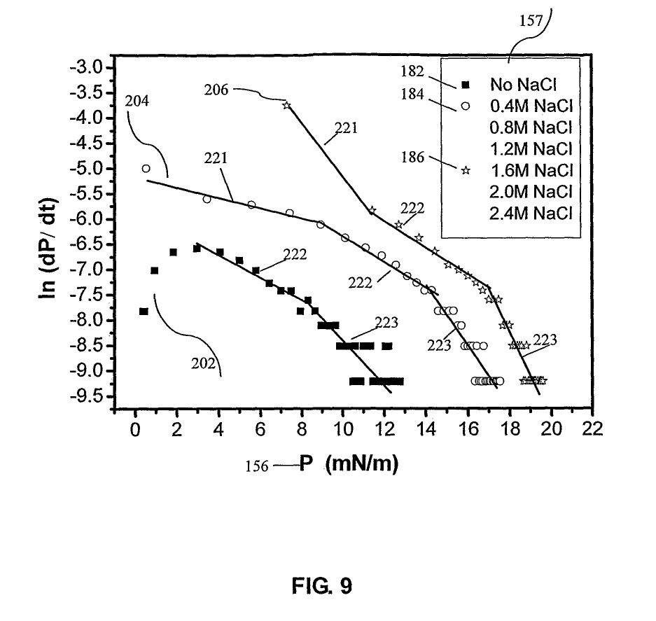

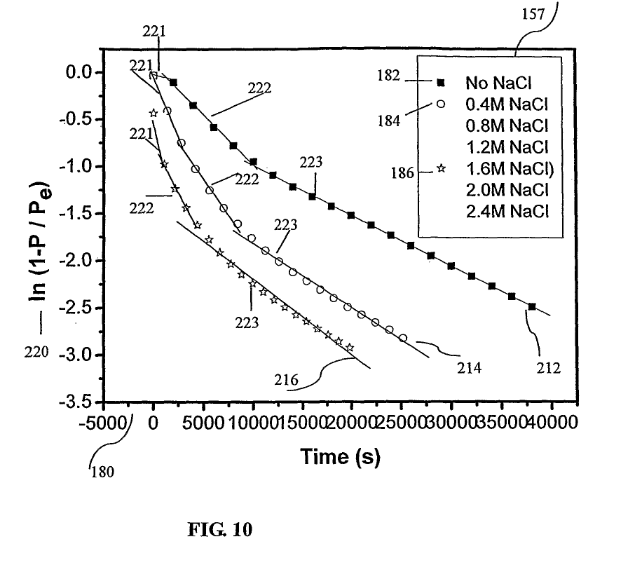

In the particular application, and without limitation to the scope of the invention, the experiment was carried out at the room temperature of 23 C. In FIGS. 4 and 5 the biomacromolecule 130 concentration is taken 1 mg/ml of lysozyme, the pH quantity is 4.5 and the buffering agent is 50 mM sodium acetate. In FIGS. 7-10 the additive is the salt NaCl, and the variable quantity 158, taken to be the additive concentration 157, is varied from 0 to 2.4 M in steps of 0.4 M. In FIGS. 7 and 8 the assembly parameter 164 is taken to be the surface tension s. In FIGS. 9 and 10 the assembly parameter 164 is the surface pressure p=s−s₀, where s₀ is the surface tension of the solvent in the absence of biomacromolecules. For clarity, and without limitation to the scope of the invention, in FIGS. 7-10 only three values of the additive concentration 157 are used to illustrate the second embodiment of the invention: 0.0 M NaCl 182, 0.4 M NaCl 184 and 1.6 M NaCl 186.

FIG. 7 shows plots 191, 192, 193 of the assembly parameter 164 taken to be the surface tension s, against time 180, for, respectively, the three additive concentrations 182, 184 and 186, taking the additive to be the salt sodium chloride (NaCl). We see that the corresponding assembly parameters 164, taken to be the surface tensions, remain unchanged when equilibrium times t_{eq} 194, 195 and 196 are reached. Each equilibrium time t_{eq} corresponds to an equilibrium surface tension Seq.

FIG. 8 shows plots 197, 198, 199 of the assembly parameter 164 against the square root of the time 180 for, respectively, the three additive concentrations 182, 184, 186. FIG. 9 shows plots 202, 204, 206 of the logarithm of the time derivative of the assembly parameter (in dp/dt) 164, taken to be the surface pressure p, against the surface pressure p, for, respectively, the three additive concentrations 182, 184, 186. FIG. 10 illustrates time-dependence profile 220 of the macromolecule behavior against time 180, which expression in the present application is given by ln(1−p/p_{eq}), where ln is the natural logarithm, p is the surface pressure and p_{eq} is an equilibrium surface pressure. The equilibrium surface pressure is given by p_{eq}=s_{eq}−s₀, where s_{eq} is the equilibrium surface tension.

In plots 212, 214, 216 of the configurational rearrangement expression 220 the equilibrium surface pressures p_{eq} are equal to the surface pressure evaluated, respectively, at the equilibrium times t_{eq} 194, 195, 196 in FIG. 7.

We notice that each plot in FIGS. 8, 9 and 10 exhibits three line segments identifying the steps 142 144 and 146 in FIGS. 4 and 5: a first straight line segment 221 identifies the diffusion step 142, a second straight line segment 222 identifies the penetration step 144, and a third straight line segment 223 identifies the rearrangement step 146. In FIG. 10 the diffusion time t_{diff} is calculated by the inverse of the slope of the first straight line segment 221, the penetration time tₚₑₙ is calculated by the inverse of the slope of the second straight line segment 222, and the rearrangement time tₐᵣᵣ is calculated by the inverse of the slope of the third straight line segment 223, so that the integration time is obtained as a sum of the last two, tᵢₙₜ=tₚₑₙ+tₐᵣᵣ.

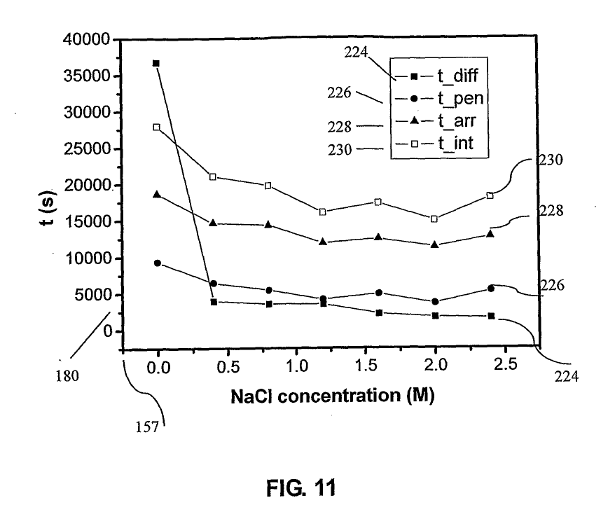

FIG. 11 shows a plot 224 of the diffusion time t_{diff}, a plot 226 of the penetration time tₚₑₙ, a plot 228 of the rearrangement time tₐᵣᵣ, and a plot 230 of the integration time tᵢₙₜ=tₚₑₙ+tₐᵣᵣ, against the additive concentration 157. In this experiment the additive concentration 157 is taken in a range from 0 to 2.4 M NaCl in steps of 0.4 M NaCl. A histogram in FIG. 12 illustrates how the crystallization coefficients 232 relate to the aggregation boundary condition. The crystallization coefficients 232 k_{cryst}=tᵢₙₜ/t_{diff}, are calculated for all pairs of obtained values for the integration time tᵢₙₜ and the diffusion time t_{diff}, and are shown against the additive concentration 157 in the above range.

Crystallization Window

Subsequently crystallization experiments were carried out in order to quantify the aggregation boundary condition in terms of the crystallization coefficient k_{cryst} 232 in FIG. 12. The experimental conditions used included the aforementioned range of the additive concentration 157 (FIGS. 11 and 12) as well as the remaining applied experimental conditions referring, e.g. to pH and temperature. The biomacromolecule concentration used for the purpose of crystallization was allowed to vary. The crystallization results are classified in three domains 234 listed below and included in FIG. 12, where the crystallization coefficient 232 is shown against the additive concentration 157.

Domain A: The crystallization coefficient k_{cryst} 232 is below approximately 4.0. Neither crystallization nor aggregation occurs in the entire range of the biomacromolecule concentrations used for crystallization, at the applied experimental conditions, e.g. additive concentration 157.

Domain B: The crystallization coefficient k_{cryst} 232 is above approximately 4.0 and below approximately 8.5. Crystallization occurs for certain biomacromolecule concentrations, at the applied experimental conditions, e.g. the additive concentration 157.

Domain C: The crystallization coefficient k_{cryst} 232 is above approximately 8.5. Only aggregation, but no crystallization, occurs at the applied experimental conditions, e.g. additive concentration 157, regardless of the biomacromolecule concentrations used for crystallization.

Thus the aggregation indicator 232 employed to define the aggregation boundary condition in FIG. 12 lies below approximately 8.5, and the crystallization window is approximately given by the range 4.5<k_{cryst}<8.5. An empirical result of our experiments is that in practice, a diffusion time that is short enough to enable crystallizing particles to approach one another and group together is reflected in a minimum value of k_{cryst}=4 or 4.5. That means, that below about K_{cryst}=4 or 4.5 crystallization is usually not observed. The value of the crystallization coefficient indicating the onset of amorphous aggregation is the upper bound, empirically found to lie around K_{cryst}=8.5 or 9.

### EXAMPLE 1

### Experimental Result from Solution of the Protein Lysozyme in Sodium Chloride

A combination of the crystal equilibrium condition in FIG. 1A or FIG. 6 and the aggregation boundary condition in FIG. 1B or FIG. 12 are used to illustrate how crystallization and aggregation conditions can be predicted. As regards the crystal equilibrium condition, the point 171 on the solubility curve 170 of FIG. 1A or FIG. 6 corresponds to a pair of the additive (salt) concentration 157 that equals 1 M NaCl), and the biomacromolecule (lysozyme) concentration 156 (that equals 4 mg/ml). The crystal equilibrium condition is prescribing that crystallization can occur when the salt and lysozyme concentrations are taken above this pair of values. Thus the equilibrium lysozyme concentration equals 4 mg/ml when the additive concentration 157 is 1 M NaCl. The crystallization lysozyme concentration must therefore exceed 4 mg/ml.

As regards the aggregation boundary condition, FIG. 1B or FIG. 12 shows that when the salt concentration 157 is taken above around 1.9 M NaCl, no crystallization may be expected to occur, regardless of the lysozyme concentration used for crystallization; in other words any lysozyme concentration used for crystallization in this case has a very high probability of resulting in the amorphous aggregation. The crystallization coefficient k_{cryst} 232 associated with the pair: salt concentration 157 equal to 1 M NaCl, lysozyme concentration 156 equal to 1 mg/ml in FIG. 1B or FIG. 12 is approximately 4.8. Therefore, when the salt concentration is 1 M NaCl, it should be possible for the crystallization to take place. That means that it should be possible to find some range of lysozyme concentration to use for the purpose of successful crystallization. Indeed, the crystallization does occur when the lysozyme concentration used for crystallization in this case is above 20 mg/ml.

### EXAMPLE 2

### Experimental Result from Solution of Protein Lysozyme in Ammonium Sulfate

FIG. 13 shows a plot 240 of the assembly parameter 164 taken to be the surface tension s, against time 180, when the biomacromolecule solution 242 includes the protein lysozyme and the additive salt ammonium sulfate (NH₄)₂SO₄. The biomacromolecule concentration 156 is 1 mg/ml, and the additive concentration 157 is 2 M. This experiment resulted in the following values for the diffusion time, the penetration time, the rearrangement time, and the integration time: t_{diff}=833.33 sec, tₚₑₙ=2320 sec, tₐᵣᵣ=5537 sec, tᵢₙₜ=7857 sec. The corresponding value of the crystallization coefficient 232 k_{cryst} in FIG. 12 was 9.43. According to the aggregation boundary condition established in the present method, crystallization experiments using the aforementioned lysozyme and ammonium sulfate concentrations should lead to the amorphous aggregation 128 in FIG. 3. Reports in the scientific literature confirm our result that lysozyme cannot be crystallized from the ammonium sulfate solution. However use of the method employing the second virial coefficient B₂₂ leads to the incorrect prediction that the crystallization 126 in FIG. 2 of lysozyme from ammonium sulfate should be possible.

### EXAMPLE 3

### Experimental Result from Solution of Protein Concanavalin A in Ammonium Sulfate

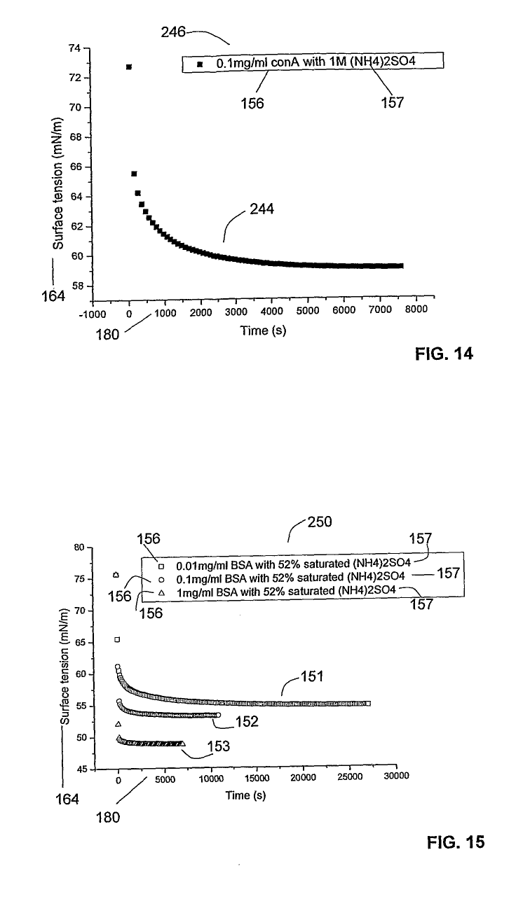

FIG. 14 shows a plot 244 of the assembly parameter 164 taken to be the surface tension s, against time 180, when the biomacromolecule solution 246 includes the protein concanavalin A (about 23 kdalton) and the additive salt ammonium sulfate (NH₄)₂SO₄. The biomacromolecule concentration 156 is 0.1 mg/ml and the additive concentration 157 is 1 M. This experiment resulted in the following values for the diffusion time, the penetration time, the rearrangement time, and the integration time: t_{diff}=277.78 sec, tₚₑₙ=1436.78 sec, tₐᵣᵣ=675.67 sec, tᵢₙₜ=2112.45 sec. The corresponding value of the crystallization coefficient 232 k_{cryst} in FIG. 12 was 7.605. According to the aggregation boundary condition established in the present method, crystallization experiments using the aforementioned concanavalin A and ammonium sulfate concentrations should lead to the crystallization 126 in FIG. 2. Reports in the scientific literature confirm our result that concanavalin A does indeed crystallize from the ammonium sulfate solution 246. No predictions based on the method employing the second virial coefficient B₂₂ are available in the literature.

### EXAMPLE 4

### Experimental Result from Solution of Protein Bovine Serum Albumin in Ammonium Sulfate

FIG. 15 shows plots 251, 252, 253, of the assembly parameter 164 taken to be the surface tension s, against the time 180. The biomacromolecule solution 250 includes the protein Bovine Serum Albumin (BSA, 66 kdalton) and the additive salt ammonium sulfate (NH₄)₂SO₄. The biomacromolecule concentrations 156 are, respectively, 0.01 mg/ml, 0.1 mg/ml 254 and 1.0 mg/ml, and the additive concentration 157 is 52% saturated ammonium sulfate. This experiment resulted in the following corresponding values of the crystallization coefficient 232 k_{cryst} in FIG. 12: 24.6, 24.8 and 23.8. According to the aggregation boundary condition established in the present method, crystallization experiments using the aforementioned BSA and ammonium sulfate concentrations should lead to the amorphous aggregation 128 in FIG. 3. Reports in the scientific literature however indicate that crystallization does indeed occur. This discrepancy is attributed to the tendency of BSA to dissociate and unfold on the surface 152 of the solution 120 in FIG. 4, i.e. in this case the solution/air interface. As a consequence of this tendency, the surface tension measurements prescribed in the present method offer an unreliable picture of the BSA crystallization window.

### CONCLUSION

The prediction of the crystallization window through the embodiments of the present invention features the crystal equilibrium condition, with reference to FIGS. 1A and 6, and the aggregation boundary condition, with reference to FIG. 1B and FIG. 12. Both embodiments are applicable for biomacromolecules that are not prone to severe unfolding at the surface 152 of the solution 120 (FIG. 4). The crystal equilibrium condition follows from the biomacromolecule solubility curve 170 (FIG. 6), and defines the lower bound of the biomacromolecule concentration to be used for crystallization.

The boundary aggregation condition refers to the solvent experimental parameters. It is applicable when the biomacromolecule solutions used to determine the crystallization coefficient k_{cryst} 232 (FIG. 12) are dilute. The results are summarized for various experiments in Table 1, where the employed biomacromolecules are proteins. The first column lists the protein concentrations 156 in FIGS. 7-15 used for predicting the crystallization window by determining the aggregation boundary condition. The protein concentrations used for the crystallization in the case of lysozyme are allowed to vary in a range 5-100 mg/ml (not reported in Table 1). The second column lists the salt concentration 157 (FIGS. 7-15) used for determining the aggregation boundary condition, and also featuring in the crystallization experiment. The third column lists the pH, and the fourth column lists the resulting crystallization coefficient 232 k_{cryst} (FIG. 12). The fifth column lists the outcome of the crystallization experiments in terms of the domains A, B or C, as predicted by the aggregation indicator of the present method. These predictions are compared with the predictions obtained from the method used in the prior art employing the second virial coefficient B₂₂. Both methods are compared with experimental results in the last two columns of Table 1, whenever such results are available.

TABLE 1

| Protein, | Salt, | | | Crystallization | | |
|---|---|---|---|---|---|---|
| concentration | concentration | | | result, | B₂₂ | Surface tension |
| (mg/ml) | (M) | pH | k_{cryst} | domain (FIG. 12) | prediction | prediction |
| lysozyme, | sodium chloride, | 4.5 | 0.8 | solution, A | correct | correct |
| 1.0 | <0.3 | | | | | |
| lysozyme, | sodium chloride, | 4.5 | 4-7 | crystal, B | correct | correct |
| 1.0 | 0.3-2.0 | | | | | |
| lysozyme, | sodium chloride, | 4.5 | 8-12 | aggregation, C | correct | correct |
| 1.0 | >2.0 | | | | | |
| lysozyme, | ammonium sulfate, | 4.5 | 9.5 | aggregation, C | incorrect | correct |
| 1.0 | 2.0 | | | | | |
| concalavine A, | ammonium sulfate, | 7.0 | 7.6 | crystal, B | unavailable | correct |
| 0.1 | 1.0 | | | | | |
| BSA, | ammonium sulfate, | 6.2 | 24 | aggregation, C | unavailable | incorrect |
| 0.01, 0.1, 1.0 | 52% saturated | | | | | |

We see from Table 1 that the aggregation boundary condition in FIG. 12 prescribes that when the crystallization coefficient 232 is larger than approximately 8.5, the biomacromolecule has a high probability to undergo the amorphous aggregation 128 in FIG. 3 at the employed experimental conditions involving, e.g., specifications of solvent, additive, their concentrations or the pH. Hence the aggregation indicator defines successfully a relatively narrow crystallization window 4.5<k_{cryst}<8.5, or a broader crystallization window 4.0<k_{cryst}<9.0, for biomacromolecules, with the following provisions: first that the biomacromolecules are able to maintain their conformations at the solution/air interface; and, second, relatively low biomacromolecule concentrations, e.g. not exceeding approximately 1 mg/ml, are used in the prescribed experiments to determine the aggregation boundary condition.

1. A method for predicting a crystal equilibrium condition for biomacromolecule crystallization and for crystallizing a biomacromolecule, comprising:
setting up at least one biomacromolecule solubility experiment, comprising the steps of:
a) preparing a solution of the biomacromolecule in a solvent, the solution having a biomacromolecule concentration;
b) selecting a variable quantity;
c) selecting an assembly parameter, being one or more of a surface tension and a surface pressure;
d) monitoring a response of the assembly parameter while varying the variable quantity in a suitable way so that the response exhibits a transition;
e) obtaining an equilibrium biomacromolecule concentration based on the transition; and
f) defining a crystal equilibrium condition according to which a biomacromolecule crystallization concentration exceeds the equilibrium biomacromolecule concentration,
and crystallizing the biomacromolecule.

2. The method of claim 1, wherein the solution has further a pH and a temperature, and the variable quantity is one of the biomacromolecule concentration, the pH and the temperature.

3. The method of claim 2, wherein the solution further comprises an additive, the solution has an additive concentration, and the variable quantity is one of the biomacromolecule concentration, the pH, the temperature and the additive concentration.

4. The method of claim 1, wherein the solution has a surface.

5. The method of claim 4, wherein the biomacromolecule is not prone to unfolding at the surface of the solution.

6. The method of claim 2, wherein the transition is associated with a critical magnitude of the variable quantity.

7. The method of claim 2, wherein the transition is between a changing response of the assembly parameter and a substantially unchanging response of the assembly parameter.

8. The method of claim 2, wherein the transition is associated with a critical magnitude of the variable quantity, and further wherein the transition is between a changing response of the assembly parameter and a substantially unchanging response of the assembly parameter.

9. The method of claim 8, wherein the substantially unchanging response corresponds to a substantially minimal value of the assembly parameter.

10. The method of claim 8, further defining the crystal equilibrium condition in terms of the critical magnitude, wherein the crystal equilibrium condition prescribes that no crystallization occurs when the variable quantity is smaller than the critical magnitude.

11. The method of claim 10, wherein the variable quantity is the biomacromolecule concentration, and consequently the equilibrium biomacromolecule concentration equals the critical magnitude.

12. The method of claim 10, wherein the variable quantity is not the biomacromolecule concentration, and consequently the equilibrium biomacromolecule concentration equals the biomacromolecule concentration.

13. The method of claim 1, wherein the biomacromolecule to be crystallized is a protein.

14. The method of claim 13, wherein the protein has a weight less than 200 kDalton.

15. The method of claim 14, wherein the protein is one of a lysozyme and a concanavalin A.

16. The method of claim 1, wherein the biomacromolecule to be crystallized is a polypeptide.

17. The method of claim 1, wherein the biomacromolecule to be crystallized is a nucleic acid.

18. The method of claim 1, wherein the biomacromolecule to be crystallized is a virus.

19. The method of claim 1, wherein the biomacromolecule to be crystallized is a virus fragment.

20. The method of claim 3, wherein the additive is a salt.

21. The method of claim 3, wherein the additive comprises organic molecules.

22. The method of claim 3, wherein the additive comprises polymers.

23. A method for predicting a crystal equilibrium condition for protein crystallization and for crystallizing a protein, comprising
setting up at least one biomacromolecule solubility experiment, comprising the steps of:
a) preparing a solution of the protein in a solvent, the solution further comprising an additive, the solution having a protein concentration, an additive concentration, a pH and a temperature, the solution having a surface, the surface having a surface tension and a surface pressure, the protein being not prone to unfolding at the surface;
b) defining an assembly parameter to be one of the surface tension and the surface pressure;
c) selecting a first variable quantity and a second variable quantity from the protein concentration, the additive concentration, the pH and the temperature;
d) varying the first variable quantity in a suitable way so that the assembly parameter exhibits a transition between a changing response and a substantially unchanging response, wherein the substantially unchanging response corresponds to a first substantially minimal value of the assembly parameter, the transition being associated with a first critical magnitude of the first variable quantity;
e) varying the second variable quantity in a suitable way so that the assembly parameter exhibits a transition between a changing response and a substantially unchanging response, wherein the substantially unchanging response corresponds to a second substantially minimal value of the assembly parameter, the transition being associated with a second critical magnitude of the second variable quantity;
f) constructing a solubility curve comprising points, each point being a pair of the first critical magnitude and the second critical magnitude, in order to assist in defining a crystal equilibrium condition; and
g) obtaining an equilibrium protein concentration and defining the crystal equilibrium condition which is based on the solubility curve, and which prescribes that crystallization occurs when the first variable quantity exceeds the first critical magnitude of the pair, and the second variable quantity exceeds the second critical magnitude of the pair,
and crystallizing the protein using a protein crystallization concentration exceeding the equilibrium protein concentration.

24. The method of claim 23, wherein in step (c) the protein concentration is one of the first variable quantity and the second variable quantity, and hence in step (g) the equilibrium protein concentration is correspondingly one of the first critical magnitude and the second critical magnitude.

25. The method of claim 23, wherein in step (c) the protein concentration is not one of the first variable quantity and the second variable quantity, and hence in step (g) the equilibrium protein concentration is the protein concentration.

26. The method of claim 23, wherein the protein is one of a lysozyme and a concanavalin A and the additive is a salt.

27. A method for predicting an aggregation boundary condition for biomacromolecule crystallization and for crystallizing a biomacromolecule, comprising:
setting up at least one aggregation boundary condition experiment comprising:
a) preparing a solution of the biomacromolecule;
b) selecting a variable quantity;
c) selecting an assembly parameter being one or more of a surface tension and a surface pressure;
d) measuring the assembly parameter at different times;
e) registering an equilibrium assembly parameter;
f) deriving a crystallization coefficient from the equilibrium assembly parameter, the crystallization coefficient being associated with the variable quantity; and
g) using an aggregation indicator to define an aggregation boundary condition for the biomacromolecule, the aggregation boundary condition prescribing that an aggregation occurs when the crystallization coefficient associated with the variable quantity is larger than the aggregation indicator,
and crystallizing the biomacromolecule.

28. A method for predicting an aggregation boundary condition for biomacromolecule crystallization and for crystallizing a biomacromolecule, comprising:
setting up at least one aggregation boundary condition experiments comprising:
a) preparing a solution of the biomacromolecule in a solvent, the solution having a biomacromolecule concentration and a surface, the surface having a surface pressure;
b) selecting a variable quantity;
c) obtaining the surface pressure at different times, while varying the variable quantity;
d) recording a time dependent equilibrium surface pressure which is associated with the variable quantity:
e) formulating a time-dependence profile based on the equilibrium surface pressure, which is associated with the variable quantity;
f) deriving from the time-dependence profile a crystallization coefficient of the biomacromolecule, that is associated with the variable quantity; and
g) obtaining from the crystallization coefficient an aggregation indicator in order to define an aggregation boundary condition for the biomacromolecule, the aggregation boundary condition prescribing that an aggregation occurs when the crystallization coefficient associated with the variable quantity is larger than the aggregation indicator,
and crystallizing the biomacromolecule.

29. The method of claim 28, wherein the biomacromolecule is not prone to unfolding at the surface of the solution.

30. The method of claim 28, wherein the solution further has pH and a temperature.

31. The method of claim 28, wherein the biomacromolecule concentration is in the range 0.01-1.2 mg/ml.

32. The method of claim 28, wherein the solution further comprises an additive and the solution has an additive concentration.

33. The method of claim 30, wherein the variable quantity is one of the biomacromolecule concentration, the pH and the temperature.

34. The method of claim 32, wherein the variable quantity is one of the biomacromolecule concentration, the additive concentration, the pH and the temperature.

35. The method of claim 28, wherein the step of deriving the crystallization coefficient comprises the steps of:
obtaining a diffusion time of the biomacromolecule;
obtaining an integration time of the biomacromolecule; and
dividing the integration time by the diffusion time to obtain the crystallization coefficient of the biomacromolecule, that is associated with the variable quantity.

36. The method of claim 28 wherein the time-dependence profile is given by ln(1−p/p_{eq}), where ln is the natural logarithm, p is the surface pressure and p_{eq} is an equilibrium surface pressure.

37. The method of claim 36, where the step of deriving the crystallization coefficient comprises the steps of:
constructing a plot of the time-dependence profile against a time;
identifying on the plot of the time-dependence profile a first substantially straight linear segment, a second substantially straight linear segment and a third substantially straight linear segment, where the second substantially straight linear segment is later in the time than the first substantially straight linear segment and the second substantially straight linear segment is later in the time than the third substantially straight linear segment;
equating a diffusion time to an inverse slope of the first substantially straight linear segment;
equating a penetration time to an inverse slope of the second substantially straight linear segment;
equating a rearrangement time to an inverse slope of the third substantially straight linear segment;
adding the penetration time and the rearrangement time to obtain an integration time; and
dividing the integration time by the diffusion time to obtain the crystallization coefficient of the biomacromolecule, that is associated with the variable quantity.

38. The method of claim 28, wherein the biomacromolecule to be crystallized is a protein.

39. The method of claim 38, wherein the protein has a weight less than 200 kDalton.

40. The method of claim 39, wherein the protein is one of a lysozyme and a concanavalin A.

41. The method of claim 28, wherein the biomacromolecule to be crystallized is a polypeptide.

42. The method of claim 28 wherein the biomacromolecule to be crystallized is a nucleic acid.

43. The method of claim 28, wherein the biomacromolecule to be crystallized is a virus.

44. The method of claim 28, wherein the biomacromolecule to be crystallized is a virus fragment.

45. The method of claim 32, wherein the additive is a salt.

46. The method of claim 32, wherein the additive comprises organic molecules.

47. The method of claim 32, wherein the additive comprises polymers.

48. The method of claim 28, wherein the aggregation indicator is below 9.

49. The method of claim 28, wherein the aggregation indicator is below 8.5.

50. The method of claim 28, wherein the aggregation indicator is in a range from 4 to 9.

51. The method of claim 28, wherein the aggregation indicator is in a range from 4.5 to 8.5.

52. A method for predicting an aggregation boundary condition for protein crystallization and for crystallizing a protein, comprising:
setting up at least one aggregation boundary condition experiment, comprising:
a) preparing a solution of the protein in a solvent, a salt, and a suitable buffer, the solution having a salt concentration, a protein concentration in a range 0.01-1.2 mg/ml, a pH and a temperature, the solution having a surface, the surface having a surface pressure, the protein not being prone to unfolding at the surface of the solution;
b) obtaining the surface pressure at different times, while varying the salt concentration;
c) recording a time-dependent equilibrium surface pressure, which corresponds with an equilibrium time, and which is associated with the salt concentration;
d) formulating a time-dependence profile, which is given by ln(1−p/p_{eq}), where ln is the natural logarithm, p is the surface pressure and p_{eq} is an equilibrium surface pressure, and which is associated with the salt concentration;
e) constructing a plot of the time-dependence profile against a;
f) identifying on the plot a first substantially straight linear segment, a second substantially straight linear segment and a third substantially straight linear segment, where the second substantially straight linear segment is later in the time than the first substantially straight linear segment, and the third substantially straight linear segment is later in time than the second substantially straight linear segment;
g) equating a diffusion time to an inverse slope of the first substantially straight linear segment;
h) equating a penetration time to an inverse slope of the second substantially straight linear segment;
i) equating a rearrangement time to an inverse slope of the third substantially straight linear segment;
j) adding the penetration time and the rearrangement time to obtain an integration time;
k) dividing the integration time by the diffusion time to obtain the crystallization coefficient of the protein, that is associated with the salt concentration, concentration;
g) obtaining from the crystallization coefficient an aggregation indicator in order to define an aggregation boundary condition for the protein, the aggregation boundary condition prescribing that an aggregation occurs when the crystallization coefficient associated with the salt concentration is larger than the aggregation indicator, the aggregation indicator being in a range from 4.5 to 8.5.

53. The method of claim 52, wherein the protein is one of a lysozyme and a concanavalin A.

54. The biomacromolecule crystallized by the method of claim 1.

55. The protein crystallized by the method of claim 25.

56. The biomacromolecule crystallized by the method of claim 28.

57. The protein crystallized by the method of claim 52.

58. The method of claim 3, wherein the transition is associated with a critical magnitude of the variable quantity.

59. The method of claim 3, wherein the transition is between a changing response of the assembly parameter and a substantially unchanging response of the assembly parameter.

60. The method of claim 3, wherein the transition is associated with a critical magnitude of the variable quantity, and further wherein the transition is between a changing response of the assembly parameter and a substantially unchanging response of the assembly parameter.
